(19) United States
(12) Patent Application Publication
(10) Pub. No.: US 2022/0148570 A1
(43) Pub. Date: May 12, 2022

(54) SPEECH INTERPRETATION DEVICE AND SYSTEM

(71) Applicants: TECHNOLOGIES OF VOICE INTERFACE LTD., Ramat Ga (IL); SCJ ASSOCIATES, INC., ROCHESTER, NY (US)

(72) Inventors: DANNY LIONEL WEISSBERG, RAMAT GAN (IL); STEVEN H. GRIFFITH, HONEOYE FALLS, NY (US); MERRY RIEHM-CONSTANTINO, BUFFALO, NY (US); AMY BETH HANGEN, CLARENCE, NY (US); SCOTT S. SUTHERLAND, ROCHESTER, NY (US); RICHARD FIGUERAS, EAST ROCHESTER, NY (US)

(21) Appl. No.: 17/433,836

(22) PCT Filed: Feb. 24, 2020
(86) PCT No.: PCT/IB2020/051530

§ 371 Date: Aug. 25, 2021

Related U.S. Application Data
(60) Provisional application No. 62/809,778, filed on Feb. 25, 2019.

(51) Int. Cl. G10L 15/06; G10L 15/26; G10L 13/08; G10L 25/78

(52) U.S. Cl. CPC G10L 15/063; G10L 25/78; G10L 13/08; G10L 15/26

(57) ABSTRACT

According to a first aspect of the present disclosed subject matter a speech interpretation device designed to be worn by a user, the device comprising: at least one voice input component configured to acquire unintelligible spells the by the user; a processor coupled with memory comprising speech recognition module configured to recognize the unintelligible-spell and associate it with one of a plurality of intelligible-spells; and at least one speaker configured to play the intelligible-spell. The unintelligible spells are probes by the user during a recognition mode of the device and exemplars the by the user during a training mode of the device.

### CROSS-REFERENCE TO RELATED APPLICATION

This application claims priority under 35 U.S.C. § 119(e) from co-pending; U.S. Provisional Patent Application No. 62/809,778, by Danny Weissberg, titled “Speech Recognition Device”, filed on Feb. 25, 2019, which is incorporated in its entirety by reference for all purposes.

### TECHNICAL FIELD

The present disclosed subject matter relates to voice communication systems. More particularly, the present disclosed subject matter relates to interpretation of distorted talk caused by speech disorders.

### BACKGROUND

Millions of people around the world suffer from speech disorders manifested in distorted talk resulting from, but not limited to, the following speech disorders:

a. Inconsistent production of sounds and rearranging of sounds in a word, i.e. apraxia of speech;

b. Cluttering, characterized by the rapid rate of speech making it difficult to understand;

c. Developmental verbal dyspraxia, i.e. childhood apraxia of speech;

d. Weakness or paralysis of speech muscles caused by nerves damage, brain damage, head or neck injuries, or cerebral palsy;

e. Alterations in intensity, the timing of utterance segments, rhythm, and intonation of words;

f. Difficulty in producing specific speech sounds (phonetic disorders) resulting from the difficulty of learning to produce sounds, mainly attributed to the hearing-impaired;

g. Larynx functionality and vocal resonance;

h. Stuttering.

It should be noted that a substantial amount of people suffering from speech disorders also have a motoric disability that can impact their ability to use other communication means, such as a keyboard, or the like.

One solution to speech disorders is provided in U.S. Pat. No. 9,754,580, by Weissberg et al., titled “System and method for extracting and using prosody features”, granted on Sep. 5 2017. The system and method include an arrangement for acquiring an input voice, a signal processing library for extracting acoustic and prosodic features of the acquired voice, a database for storing a recognition dictionary, at least one instance of a prosody detector for carrying out a prosody detection process on extracted respective prosodic features, communicating with an end-user application for applying control thereto.

### BRIEF SUMMARY

According to a first aspect of the present disclosed subject matter a speech interpretation device designed to be worn by a user, the device comprising: at least one voice input component configured to acquire unintelligible spells the by the user; a processor coupled with memory comprising speech recognition module configured to recognize the unintelligible-spell and associate it with one of a plurality of intelligible-spells; and at least one speaker configured to play the intelligible-spell.

In some exemplary embodiments, the unintelligible spells are probes the by the user during a recognition mode of the device and exemplars the by the user during a training mode of the device.

In some exemplary embodiments, the at east one voice input component is selected from a group consisting of: directional microphones; omnidirectional microphones; laser microphones; external microphones connected via a socket; and any combination thereof.

In some exemplary embodiments, the processor and the at least one voice input component are configured for voice activation detection comprising user's voice isolation; users voice extraction and user's voice enhancement with respect to ambient sounds.

In some exemplary embodiments, the at least one voice input component is further configured to acquire intelligible-spells in the training mode.

In some exemplary embodiments, the device further comprises communication interfaces selected from a group consisting of: a Wi-Fi transceiver; a Bluetooth transceiver; and USB interface, wherein the Bluetooth transceiver and the USB interface are configured for communicating with a proxy computer, and wherein the Wi-Fi transceiver is configured for communicating with the proxy computer and the Internet.

In some exemplary embodiments, the intelligible-spell is selected from a group consisting of a text string having at least one word; a recording comprising one or more intelligible spoken words; and a combination thereof.

In some exemplary embodiments, the memory comprising a speech recognition module utilized for generating a user model for each intelligible-spell of the plurality of intelligible-spells based on at least one exemplar recorded in the training mode for each intelligible-spell.

In some exemplary embodiments, the memory further comprises a database module retaining the user models; recorded exemplars for each spell; and at least one lexicon composed of the plurality of intelligible-spells.

In some exemplary embodiments, the memory further comprises a speech processing module utilized for determining, by user models stored in the database module, a matching intelligible-spell, stored in the database module, for each acquired probe, wherein the play the intelligible-spell is playing the matching intelligible-spell.

In some exemplary embodiments, the at least one speaker is configured playing the intelligible-spell is playing a synthesized voice made of the text string of the matching intelligible-spell or a recording of the matching intelligible spoken words, wherein the text string is synthesized by the processor.

In some exemplary embodiments, the proxy computer, hosting a user's interface application, is configured as: a text string data entry tool for entering spells or select spells from a predefined menu; a sensitizer configured to make a synthesize voice from the text string of the intelligible-spell and a speaker to play the synthesized voice; a microphone adapted to acquire intelligible spoken words; a platform for building a lexicon of spells to be grouped for different scenarios; a display providing a visual representation of a waveform representing exemplars recorded in the training mode; an editor for editing recorded exemplars; and a proxy to the internet.

According to another aspect of the present disclosed subject matter a speech interpretation system comprising: the speech interpretation device; the proxy computer hosting a user's interface application and a cloud computing server (CCS) comprising: a speech recognition server; a speech pattern recognition application; and a speech recognition database; wherein the computer and the CCS comprising communication interfaces for communicating with each other and the device.

In some exemplary embodiments, the communication interfaces are selected from a group consisting of: interfaces; a Bluetooth interfaces; and USB interface, wherein the Bluetooth interfaces and the USB interface are configured for communicating between the proxy computer and the device, and wherein the interfaces are configured for communicating between the device the proxy computer and the CCS via the Internet.

In some exemplary embodiments, the speech recognition database retains at least one exemplar recorded for each spell in the training mode; and at least one lexicon composed of the plurality of intelligible-spells obtained from the device in the training mode.

In some exemplary embodiments of the disclosed subject matter, the speech recognition server and the speech pattern recognition application generate a user model for each intelligible-spell of the plurality of intelligible-spells based on at least one exemplar recorded in the training mode for each intelligible-spell, and wherein the speech recognition server retains the user model for each intelligible-spell of the plurality of intelligible-spells in the speech recognition database and the database module of the device.

In some exemplary embodiments, the system is configured to concurrently communicate and manage and support a plurality of devices used by different registered users.

In some exemplary embodiments, the communicate manage and support comprising: user registration; access control; management registration credentials; users lexicon maintenance and user models updates for the different registered user.

A training mode method for the comprising: entering at least one intelligible-spell; recording at least one exemplar for each intelligible-spell; processing the at least one exemplar; generating a user model for each intelligible-spell; and retaining the user model in the database module.

In some exemplary embodiments, the entering at least one intelligible-spell is selected from a group consisting of: typing a text string representation of the intelligible-spell by the computer; recording intelligible spoken words by the device or the computer; and a combination thereof.

In some exemplary embodiments, the processing comprising providing for editing the exemplars with the user's interface application.

According to a yet another aspect of the present disclosed subject matter a training mode method for the speech interpretation system comprising: entering, by the proxy computer, one or more intelligible-spells; recording, by the device, at least one exemplar for each intelligible-spell; retrieving, by the speech recognition server, the one or more intelligible-spells and the recording of the at least one exemplar for each intelligible-spell; processing the recording by the speech recognition server; generating, by a speech pattern recognition application, a user model for each intelligible-spell; and storing the user model in the speech recognition database and the database module of the device.

In some exemplary embodiments, the entering at least one intelligible-spell is selected from a group consisting of: typing a text string representation of the intelligible-spell by the computer; recording intelligible spoken words by the device or the computer; and a combination thereof.

In some exemplary embodiments, the processing comprising providing for editing the exemplars with the user's interface application.

According to a yet another aspect of the present disclosed subject matter a recognition mode method for the speech interpretation device comprising acquiring a probe; determining a matching intelligible-spell for the probe synthesizing the matching intelligible-spell playing a synthesized outcome of the matching intelligible-spell.

In some exemplary embodiments, the synthesizing is selected from a group consisting of synthesizing by the sensitizer of the computer; synthesizing made by the device; and a combination thereof.

In some exemplary embodiments, the playing is selected from a group consisting of: playing the synthesized outcome by the speakers of the device; playing the synthesized outcome by the speakers of the computer; and a combination thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the specification, including definitions, will control. In addition, the materials, methods, and examples are illustrative only and not intended to be limiting.

### BRIEF DESCRIPTION OF THE DRAWINGS

Some embodiments of the disclosed subject matter described, by way of example only, with reference to the accompanying drawings. With specific reference now to the drawings in detail, it is stressed that the particulars shown are by way of example and for purposes of illustrative discussion of the preferred embodiments of the present disclosed subject matter only, and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the disclosed subject matter, the description taken with the drawings making apparent to those skilled in the art how the several forms of the disclosed subject matter may be embodied in practice.

In the drawings:

FIG. 1 shows an illustration of a speech interpretation device, in accordance with some exemplary embodiments of the disclosed subject matter;

FIG. 2 illustrates a user wearing the speech interpretation device, in accordance with some exemplary embodiments of the disclosed subject matter;

FIG. 3 shows a block diagram of a system comprising the speech interpretation de in accordance with some exemplary embodiments of the disclosed subject matter;

FIG. 4 shows a flowchart diagram of a training session, in accordance with some exemplary embodiments of the disclosed subject matter; and

FIG. 5 shows a flowchart diagram of a recognition session, in accordance with some exemplary embodiments of the disclosed subject matter.

### DETAILED DESCRIPTION

Before explaining at least one embodiment of the disclosed subject matter in detail, it is to be understood that the disclosed subject matter is not limited in its application to the details of construction and the arrangement of the components set forth in the following description or illustrated in the drawings. The disclosed subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

The terms “comprises”, “comprising”, “includes”, “including”, and “having” together with their conjugates mean “including but not limited to”. The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosed subject matter may be presented in a range format. It should be understood that the description in range format is merely for convenience and brevity and should not be construed as an inflexible limitation on the scope of the disclosed subject matter. Accordingly, the description of a range should be considered to have specifically disclosed all the possible sub-ranges as well as individual numerical values within that range.

It is appreciated that certain features of the disclosed subject matter, which are, for clarity, described in the context of separate embodiments, may also he provided in combination in a single embodiment. Conversely, various features of the disclosed subject matter, which are, for brevity, described in the context of a single embodiment, may also be provided separately or in any suitable sub-combination or as suitable in any other described embodiment of the disclosed subject matter. Certain features described in the context of various embodiments are not to be considered essential features of those embodiments unless the embodiment is inoperative without those elements.

An objective f the present disclosure is providing a user having speech disorder, with a speech interpretation device. In some exemplary embodiments, the speech interpretation device is a hands-free operated device adapted to be worn on the user's neck or shoulders.

In some exemplary embodiments of the disclosed subject matter, the speech interpretation device comprises voice input components, such as microphones, configured to acquire unintelligible phrases and/or words, i.e. distorted talk, spoken by the user.

In some exemplary embodiments, the speech interpretation device is configured to interpret the distorted talk into intelligible phrases and/or words for one or more people that are conversing with the user. The speech interpretation device can be configured to play the intelligible phrases and/or words utilizing a built-in speaker or other communication methods to the people conversing with the user.

Additionally, or alternatively, the intelligible phrases and words may be audibly played out, or textually outputted, through an external device other than the wearable device such as an external speaker or a computing device having a speaker and/or a display screen such as a smartphone, a PC, a laptop, a tablet, and any combination thereof, or the like.

In some exemplary embodiments, wearing the device around the shoulder or neck area places the voice input components proximal to the user's mouth, thus allowing better voice resolution and sound differentiation. Additionally, the placement of the device around the shoulder or neck area facilitates operation by users having motoric disability that are unable to hold a device with their hands or unable to hold the device for an extended period of time.

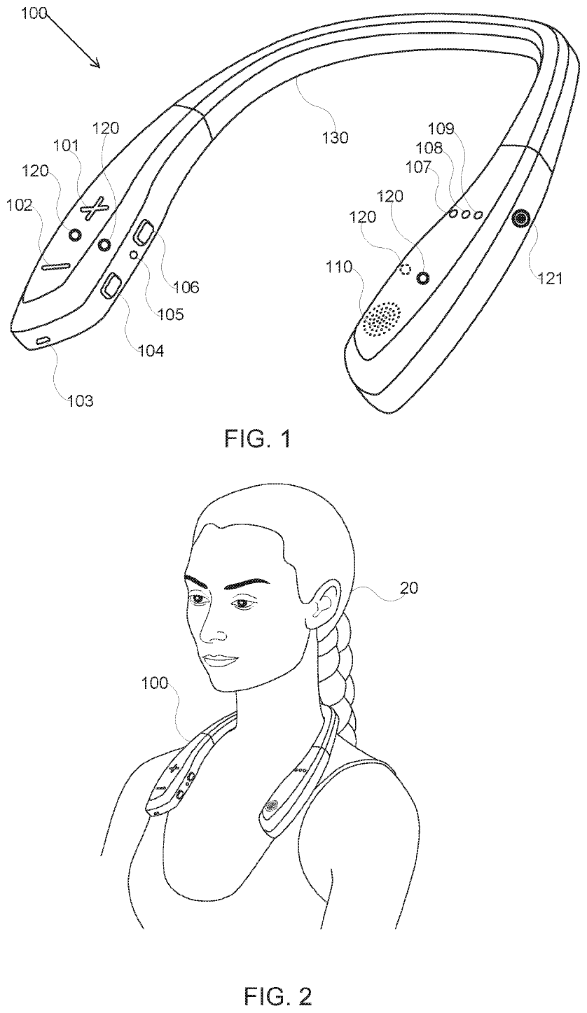

Referring now to FIG. 1 showing an illustration of a speech interpretation device, in accordance with some exemplary embodiments of the disclosed subject matter. The speech interpretation device 100 comprises a volume up button 101 and volume down button 102 for adjusting the volume of at least one speaker 110. The at least one speaker is configured to audibly output the intelligible words and/or phrases that are generated by device 100.

In some exemplary embodiments, the speech interpretation device 100 can be powered by an internal battery power supply (not shown) or an external power supply (not shown), connected via USB port 103, that can also be used for charging the battery. Additionally, or alternatively, USB port 103 may also be used as a communication interface between device 100 and external computer for setting, configuring, testing and any combination thereof, as well as outputting outcomes of the device to a computer (not shown). Furthermore, device 100 comprises an on/off power switch 104 and a power indicator 105 that reflects the battery power status.

In some exemplary embodiments, device 100 comprises a communication activation push-button 106 used for both Bluetooth pairing or Wi-Fi enabling/disabling. The communication activation is coupled with a Bluetooth indicator 107 and a Wi-Fi indicator 109 that reflects the Bluetooth and Wi-Fi use, i.e. Bluetooth pairing status and communication status.

In some exemplary embodiments, the speech interpretation device 100 combines a plurality of voice input components (MIC) 120 comprising directional microphones (DMIC), omnidirectional microphones (OMIC), laser microphones (LMIC), and any combination thereof, or the like. Additionally, or alternatively, the speech interpretation device 100 is configured to receive voice input from an external microphone (EMIC) connected via socket 121. All in all, voice input components are configured to isolate, extract and enhance a user's voice out of ambient sounds.

It should be noted that the speech interpretation device 100 is made of flexible material, however, the device can be provided with a set of replaceable neckbands 130 to fit different neck sizes. In some exemplary embodiments, the speech interpretation device 100 is equipped with indicator 108 that indicates voice activation detection (VAD). It will be appreciated that indicators 105, 107, 108 and 109 can be implemented with light-emitting diodes (LED).

Referring now to FIG. 2. illustrating a user wearing the speech interpretation device, in accordance with some exemplary embodiments of the disclosed subject matter. In the exemplary embodiment, user 20 is wearing the speech interpretation device 100 around the neck to allow high quality acquiring of user's 20 speech, and to differ it from other surrounding speech and surrounding noise in addition to high quality playing out to listeners of the intelligible phrases and words.

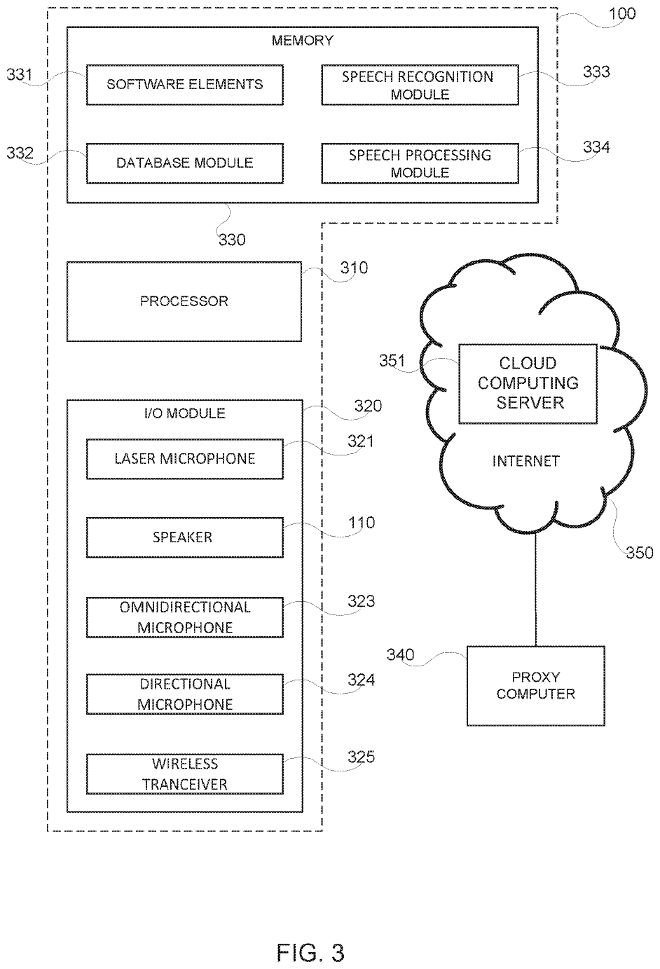

Referring now to FIG. 3 showing a block diagram of a system comprising the speech interpretation device, in accordance with some exemplary embodiments of the disclosed subject matter. In addition to the speech interpretation device 100, the system further comprises a proxy computer 340 and a cloud computing server (CCS) 351.

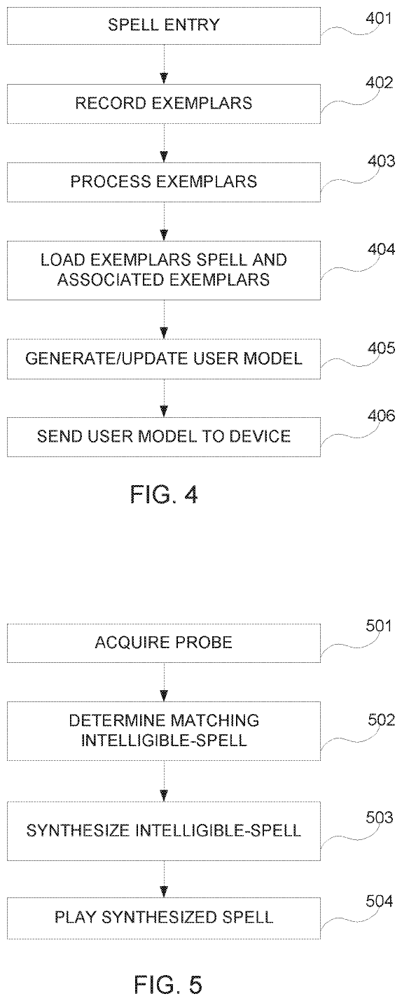

In some exemplary embodiments, the system is a speech interpretation system configured to concurrently manage and support a plurality of speech interpretation devices used by different users. Device 100, computer 340 and CCS 351 are computerized components adapted to perform methods such as depicted in FIGS. 4, and 5 and other computations associated with the operation of the system.

In some exemplary embodiments, device 100 comprises a processor 310. Processor 310 can be a central processing unit (CPU), a graphics processing unit (GPU), e.g. a processor configured for machine learning, a microprocessor, an electronic circuit, an integrated circuit (IC) or the like. Additionally, or alternatively, device 100 can be implemented as firmware written for or ported to a specific processor such as digital signal processor (DSP) or microcontrollers, or can be implemented as hardware or configurable hardware such as field-programmable gate array (FPGA) or application-specific integrated circuit (ASIC). Processor 310 can be utilized to perform computations required by device 100 or any of its subcomponents.

In some exemplary embodiments of the disclosed subject matter, device 100 comprises an Input/Output (I/O) module 320. Device 100 can utilize the I/O module 320 as an interface to transmit and/or receive information, instructions, signals and any combination thereof, or the like between processor 310 and elements that are either internal or external to device 100.

In some exemplary embodiments, I/O Module 320 can be used to provide an interface to a user 20 of the device, and his/her listeners, such as by providing outputs, visualized results, reports, and audible voice. It will be appreciated that device 100 can operate without human intervention.

In the description of the present disclosure the terms “spell” “exemplar” and “probe” refer to as follows: a spell or an intelligible-spell is composed of at least one predetermined word, i.e. a phrase, a sentence, a word, and any combination thereof. For example, “hello world”. An exemplar is a spell spoken by the user in a training session and a probe is, typically, a spell spoken by the user while conversing with others while in recognition mode. It should be noted that the exemplar and the probe are unintelligible spells spoken by the user.

The I/O Module 320 can be comprised of at least one OMIC 323, at least one DMIC 324, at least one LMIC 321, at least one speaker 110, a wireless transceiver (XCVR) 325, at least one analog to digital converter (ADC) (not shown), at least one digital to analog converter (D2A) (not shown), and a USB interface (not shown), or the like.

In some exemplary embodiments, the OMIC 323 is configured to acquire ambient sound from the user's surroundings that may also be used to catch the user's voice for more refined speech processing. Additionally, or alternatively, the OMIC 323 can be utilized, by processor 310, for detecting a direction from which the voice is coming (e.g. by energy analysis and the use of a microphone beamforming algorithm). The sound detection capability, represented by a sphere, facilitates avoidance of false signals that are not originated from the user.

In some exemplary embodiments, the DMIC 324 is configured to acquire sounds from the direction it is aimed at (the user). The DMIC 324 has a sound detection domain that can be represented by a cardioid equation, with a symmetry axis along the angle it points to. The at least one DMIC 324 is configured to acquire the user's unintelligible sound that is then converted by the ADC of the 110 module 320 into either exemplar data elements (training session) or probe data elements in the recognition mode (normal operation).

A combination of OMIC signals and the DMIC signals can be used to separate and extract the user's voice from other people's voices and/or environmental noises. Thus, providing more accurate voice activation detection (VAD). Additionally, or alternatively, device 100 can implement its recognition process for each microphone, or a group of microphones, separately, i.e. multiple recognitions for enhancing the recognition process.

In some exemplary embodiments, the LMIC 321 can be utilized for speech enhancement and/or as voice activation detection (VAD). Additionally, the I/O module 320 can also comprise an external microphone (not shown), connected via socket 121, as an optional microphone or as throat/laryngophone microphone for enhancing the VAD.

In some exemplary embodiments, the at least one speaker 100 is configured to audibly output (play) synthesized spells, i.e. intelligible words and phrases stored in the device. The synthesized spells are text data elements that undergo a digital to analog conversion, by the D2A of the I/O module 320. The volume of the speaker 110 can be controlled by volume up button 101 and volume down button 102, shown in FIG. 1.

In some exemplary embodiments, the wireless XCVR 325 is utilized for wireless communication with a proxy computer 340 and the USB interface (not shown) utilizing the USB port 103, shown in FIG. 1, for wired communication with the proxy computer 340. The wireless XCVR 325 can be provided with a Wi-Fi transceiver, a Bluetooth transceiver, and a combination thereof, or the like. However, it should be noted that the Wi-Fi transceiver is also capable of communicating directly with CCS 351 via the Internet 350.

In some exemplary embodiments, proxy computer 340 can be a smartphone, a notebook computer, a desktop computer, a tablet PC and any combination thereof, or the like. Computer 340 can be equipped with legacy communication technologies, such as WI Bluetooth, Ethernet, USB, or the like that enable connection with the Internet 350 and device 100. It will be appreciated that computer 340 can be used as a proxy that facilitates communication between the device and the CCS 351, a data entry tool for spells, a speaker, a microphone, and a user's interface platform.

In some exemplary embodiments, device 100 utilizes the wireless XCVR 325 and/or the USB interface for sending exemplars and associated spells to the CCS 351, either directly or via the proxy, for speech processing; and receiving a user model from the CCS 351 that correspond to the exemplars and spells. Additionally, the speech interpretation device 100 can utilize the wireless XCVR 325 and/or the USB interface for obtaining spells that are generated in computer 340 and send synthesized spells to be played on the computer 340. It should be noted that computer 340 comprises audio input and output components, such as microphones and speakers.

In some exemplary embodiments, the user can use the computer 340 for typing desired spells or select spells from a predefined menu of a user's interface application that runs on the computer. Optionally, the spells can be generated by voice to text conversion of a vocal input through computer 340 (or directly to device 100) by a person without a speech disorder. Ultimately, the user can generate a lexicon of spells, which may be grouped for different scenarios, for examples, spells that are appropriate for scenarios, such as restaurants, supermarkets, train stations, or the like.

In some exemplary embodiments, device 100 comprises a memory 330. Memory 330 can be persistent or volatile memory, such as a flash memory, a random-access memory (RAM), a programmable read-only memory (PROM), a re-programmable memory (FLASH), and any combination thereof, or the like. In some exemplary embodiments, memory 330 can retain program code to activate processor 310 to perform acts associated with any of the steps shown in FIGS. 4 and 5. Memory 330 can also be used to retain software elements 331, a database module 332, a speech recognition module (SRM) 333, and a speech processing module (SPM) 334 that can be implemented. as one or more sets of interrelated computer instructions, executed for example by processor 310 or by another processor. The components can be arranged as one or more executable files, dynamic libraries, static libraries, methods, functions, services, or the like, programmed in any programming language and under any computing environment.

In some exemplary embodiments, the SPM 334 can be utilized for executing a speech processing algorithm to an incoming probe to determine which spell of the user module the incoming probe matches. Optionally, the SPM can include an A/D codec and/or a D/A codec.

In some exemplary embodiments, the database module 332 is used for retaining a plurality of spells and their associated exemplars in addition to user models of the device. The memory 330 also retains a speech recognition module (SRM) 333 configured to generate the user model for all the recorded exemplars and the associated spell corresponding to an unintelligible phrase or word. Additionally, or alternatively, the speech recognition module can be retained and executed in CCS 351 or computer 340 for producing the user module for each spell and consequently communicate it to the device.

In some exemplary embodiments of the disclosed subject matter, device 100 is configured to distinguish between the user's speech and other's speech as well as environmental noise, and interpret only the user's speech either by discriminating sound inputs coming from both the DMIC and OMIC or only from the OMIC. Optionally, other microphone arrays may be configured and may include the use of external microphones. In some exemplary embodiments, processor 310 can be used for improving signal characteristics and analyzing signals, coming from any of the microphones, which will facilitate better speech recognition, improve VAD, and eliminate background noise. The unintelligible speech may be acquired by the device during recognition mode and in a training mode of operation (to be described in detail further below). In the training mode of operation, the speech interpretation device undergoes voice to data conversion to generate the exemplars associated with a spell.

At least one spell formed from intelligible words and/or phrases can be textually inputted by the user or a third person using computer 340 that will then undergo text to data to conversion to generate the spells. In training mode, the textual input can be captured utilizing a proprietary user's interface application that runs on computer 340. Any user of the present disclosure can create a lexicon (plurality) of spells for his/her device, wherein each spell is associated with specific exemplars.

In some exemplary embodiments, the system utilizes a cloud computing server (CCS) 351 deployed, in a remote location, which may be based on a collection of processing devices and services, such as the Amazon AWS cloud-computing platform. In some exemplary embodiments of the disclosed subject matter, the CCS 351 can be comprised of a speech recognition server (SRS), a speech pattern recognition application (SPRA) and a speech recognition database (SRDB).

It should be noted that the system of the present disclosure can utilize the system and operational features described in the abovementioned U.S. Pat. No. 9,754,580.

The speech recognition server (SRS) may configured to communicate with device 100 and computer 340 over the Internet and manage all SRS operations comprising: user registration and access control; receiving exemplars from the users following a training session; building and updating a user record for each registered user with the respective model applicable to the user; storing the users models, users and their associate device 100 ID details and registration credentials in the SRDB or any information associated with all registered users.

In some exemplary embodiments, the speech pattern recognition system (SPRA) is configured to determine user models from the exemplars and spells which may be retained in the device and uploaded to the SRS.

In some exemplary embodiments, XCVR 325, of device 100, is configured to communicate users' models, information associated with a registered user and exemplars of each spell with the SRS of the CCS 351. It should be noted that the user model may be sent from the SRS to device 100 when the device is first turned on, upon completion of training session; periodically for synchronization; upon a user-initiated request and any combination thereof, or the like.

In some exemplary embodiments of the disclosed subject matter, the speech interpretation device 100 comprises two modes of operation, a training mode, and a recognition mode. Typically, in the recognition mode, device 100 is configured for standalone operation, i.e. system elements (computer 340 and CCS 351) use are not required. However, in the training mode, device 100 may be configured to utilize the system elements.

In some exemplary embodiments of the disclosed subject matter, the speech interpretation device 100 can utilize its speech recognition module (SRM), while in training mode, for generating a user model, instead of the system elements.

Referring now to FIG. 4 showing a flowchart diagram of a training session, in accordance with some exemplary embodiments of the disclosed subject matter. In the training mode, the user may be provided with tools for building a lexicon that comprises a plurality of spells, wherein each spell is associated with one or more exemplars. The lexicon may be stored in the speech recognition database, of CCS 351, and/or in the database module 332 of the device and/or in computer 340.

Each user may have a lexicon which may include numerous spells (e.g. “hello”, “good morning I am hungry”, etc.). Each spell may include several exemplars (unintelligible recorded samples of the user saying the spell's word or phrase). During the training session, the user may textually add a new spell to the lexicon and then may vocally record several exemplars corresponding to the new spell. Optionally, the spell may be vocally added, by recording with device 100, one or more intelligible spells, spoken by a person not having a speech disability. In some exemplary embodiments, the user may alternatively choose to add several new exemplars to a spell that is already in the user's lexicon to increase the success rate of a voice recognition algorithm used to recognize the exemplars, thereby improving and strengthening the user model.

In some exemplary embodiments, the exemplar, spells, and models, may include metadata information that comprises parameters used by the speech recognition algorithm and/or used for data analysis. The parameter may include VAD (voice activation detection) parameters, signal timing measurement parameters (e.g. start, stop, length), physical parameters (energy, signal to noise ratio), and audio quality measurement parameters, among others.

In some exemplary embodiments, before initiating the first training session, a calibration session may be required. In the calibration session, the user may train the wearable device to determine how to interpret a given vocabulary. The device may build a model which may be saved in the device and/or at the SRS and accordingly update the user's lexicon. For example, the user may be able to choose spells to be in his or her native language, synthesized voice of a male or a female gender.

In step 401, a spell may be entered. In some exemplary embodiments, the user may utilize the user's interface application, of computer 340, for typing at least one spell, which will be trained on. The spells shall be obtained by and retained in the device.

In step 402, at least one exemplar, for each spell, may be recorded. In some exemplary embodiments, the user may be instructed, either by computer 340 display or vocally prompted by the device's speaker, to (vocally) read a spell for recording an exemplar using the microphones of the device. The reading and the recording of each spell may be repeated several times, thus creating a plurality of exemplars for each spell. The recording repetition may be necessary for mitigating variations in the unintelligible speech and/or microphone tolerances and/or any other variations in the recording process.

In step 403, at least one exemplar may be processed. In some exemplary embodiments, the recorded (signals) exemplars may be digitized by the ADC of the device, processed using the SPM 334 added to the lexicon and stored in the database module 331.

In some exemplary embodiments, the application that runs on proxy computer 340 provides the user with a visual representation of a waveform of the recorded signals (exemplars) and an editing capability for improving the recorded results. The editing (annotation) comprising setting the signal borders, i.e. start, end, recording time duration, and applying noise reduction filter. Additionally, or alternatively, the application may play the recording so the user can qualify the recording, disqualify the recording ranking (grade) the recording and any combination thereof, or the like.

In step 404, at least one processed exemplar and the spell it is associated with may be uploaded. In some exemplary embodiments, upon completion of recording and processing the exemplars for all the entered spells, device 100 upload the processed exemplar and their spells to the SRS, of CCS 351, using the XCVR 325, either directly or via computer 340.

In step 405, a user model may be generated. In some exemplary embodiments, the SPRA, of CCS 351 processes the received exemplars and their associated spells and generate a user model. The generating of user model may comprise a data augmentation process, i.e. time stretch, pitch shifting, reverberation, of recoded exemplar files, consequently extrapolating the number of exemplars beyond the number of the recorded exemplar. Thereby, improving the recognition success rate of each spell. The augmentation process can be done in real-time while training or building the user model. It should be noted that the augmentation process can be performed in the user device 100, in the proxy computer 340, or most commonly in the SRS.

In some exemplary embodiments, the SPRA and the speech recognition module 333 utilize algorithms and operational features described in the abovementioned U.S. Pat. No. 9,754,580 for generating the user model. Additionally, or alternatively, the SPRA and other elements of CCS 351 can utilize other models and algorithms of speech recognition technology for generating the user model. For example, “hidden Markov model”; “machine learning model”; “dynamic time warping” algorithms; and any combination thereof, or the like.

In step 406, the user model may be downloaded to the device. In some exemplary embodiments, the user model and optionally the exemplars and spells may be stored in the SRDB and downloaded to device 100 and/or computer 340. The user model is then used for activating the device independently for recognizing probes, the by the user, and indicate an appropriate spell, of the lexicon, to be played by the device.

Optionally, the CCS 351 may be configured to download the user model to computer 340, so the user can utilize the application for further editing, of step 403, of the exemplars to improve the recognition results. In such a case, if any of the exemplars were edited, steps 404 through 406 should be repeated.

Referring now to FIG. 5 showing a flowchart diagram of a recognition session, in accordance with some exemplary embodiments of the disclosed subject matter.

In the recognition mode, the device is configured recognizes an exemplar acquired during the training mode and may output audio and/or text of the corresponding spell. The wearable device may either continuously listen or listen upon pressing a press to talk (PTT) button (not shown) to the user's unintelligible word or phrase (probe) and, responsively, determine to which spell in the lexicon the probe is associated. A text to speech synthesizing mechanism may then be used to allow the device to generate the identified spell in a synthesized voice.

In step 501, a probe may be acquired n some exemplary embodiments, following power-on, the device consults the SRS for verifying that the user model is up to date, however previously downloaded user model may be used as a fallback. Then, device 100 utilizes its microphones for acquiring a probe followed by digitizing and processing the probe with SPM 334.

In step 502, a matching intelligible-spell may be determined. In some exemplary embodiments, processor 310 of the device uses the SPM 334 for determining matching intelligible-spell that congruous the probe, i.e. user module result. To do so, the probe is processed and analyzed by the user model to determine the highest probability ranking (matching) with any one of the intelligible-spell, stored in database module 332. If the ranking, of the user model (determination) result, is below a predetermined threshold the probe will be discarded, i.e. unintelligible words, phrases or any other sounds that don't correspond with any spell of the lexicon shall be ignored by the speech interpretation device 100. Otherwise, if the ranking of the user model result, is above a predetermined threshold the process (method) shall proceed to the next step.

In step 503, an intelligible-spell may be synthesized. In some exemplary embodiments, a text of the intelligible-spell, that matches the spell of the user model result, is obtained front database module 332 and undergo synthesizing by the D/A converter of the device.

In step 504, the synthesized spell may be played. In some exemplary embodiments, the synthesized spell is audible played out to speaker 110 of the device as an intelligible phrase or word.

In some exemplary embodiments, the proxy computer 340, connected to both CCS 350 and device 100, host a user's training application, which is a part of the user's interface application. Before using the device for the first time, the user should complete a training session that yields a user model for the user's lexicon. Afterword, the user model shall be used for recognizing the user voice saying the spells typed in the lexicon. In some exemplary embodiments, the user start building a lexicon by typing, in the application, the name of the spells he/she would like to add to the lexicon.

For example, after selecting several spells, the user starts the training by recording any spells at least 20 times. The number of sampled recordings (exemplars) is denoted, by a progress bar, in the application for each spell.

It should be noted that the purpose of the training session is to qualify the spells so that afterword they can be recognized by the device in recognition mode. Each time a user is recording a new exemplar (or a group of exemplars) the algorithm is testing if the amount of the exemplars satisfies a probability of recognizing the spell by the algorithm, i.e. spell qualification. A large number of exemplars increase the probability of successful recognition.

Additionally, or alternatively, the application is configured. to display an exemplar wave file on the proxy computer 340. The VAD indicates the quality measurement of each exemplar and whether an exemplar is qualified according to the VAD criteria. It should be noted that the spell qualification as well as building the user model are done at the server, while the recordings and the quality measurements are done on the device.

After the training session, the user has a lexicon that can be expanded, whenever the user wants to alter or add a new spell to the lexicon. In such a case, the user can enter the old/new spell in the application and. record himself/herself saying this spell as many times as needed. The recordings are done in the device 100, which is remotely controlled by the user from the application, running the proxy computer 340, which comprises control commands, such as start, stop, play, approve/discard the recording.

In some exemplary embodiments, the application that runs on proxy computer 340 provides the user with a visual representation of the waveform of the recorded signals (exemplars) and. an editing capability for improving the recorded. results. The editing (annotation) comprising setting the signal borders, i.e. start, end, recording time duration, and applying noise reduction filter. Additionally, or alternatively, the application may play the recording so the user can qualify the recording, disqualify the recording ranking (grade) the recording and any combination thereof, or the like.

Upon generating a user model by the CCS 351, the user model is automatically downloaded to device 100 so the user can activate the device independently (without involving the proxy computer 340) for recognizing probes, the by user, and indicate an appropriate spell, of the lexicon, to be played by the device. Optionally, the CCS 351 may be configured to download the user model to the computer 340, so the user can utilize the application for further editing of the exemplars to improve the recognition results.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims. All publications, patents and patent applications mentioned in this specification are herein incorporated in their entirety by reference into the specification, to the same extent as if each publication, patent or patent application was specifically and individually indicated to be incorporated herein by reference. Also, citation or identification of any reference in this application shall not be construed as an admission that such reference is available as prior art to the present invention.

1. A speech interpretation device designed to be worn by a user, the device comprising:
at least one voice input component configured to acquire unintelligible spells said by the user;
a processor coupled with memory comprising speech recognition module configured to recognize the unintelligible-spell and associate it with one of a plurality of intelligible-spells; and
at least one speaker configured to play the intelligible-spell.

2. The device of claim 1, wherein the unintelligible spells are probes said by the user during a recognition mode of the device and exemplars said by the user during a training mode of the device.

3. The device of claim 1, wherein the at least one voice input component is selected from a group consisting of: directional microphones; omnidirectional microphones; laser microphones; external microphones connected via a socket; and any combination thereof.

4. The device of claim 1, wherein the processor and said at least one voice input component are configured for voice activation detection comprising user's voice isolation; user's voice extraction and user's voice enhancement with respect to ambient sounds.

5. The device of claim 2, wherein the at least one voice input component is further configured to acquire intelligible-spells in the training mode.

6. The device of claim 1, further comprises communication interfaces selected from a group consisting of: a Wi-Fi transceiver; a Bluetooth transceiver; and USB interface, wherein the Bluetooth transceiver and the USB interface are configured for communicating with a proxy computer, and wherein the Wi-Fi transceiver is configured for communicating with the proxy computer and the Internet.

7. The device of claim 1, wherein the intelligible-spell is selected from a group consisting of a text string having at least one word; a recording comprising one or more intelligible spoken words; and a combination thereof.

8. The device of claim 2, wherein the memory comprising a speech recognition module utilized for generating a user model for each intelligible-spell of the plurality of intelligible-spells based on at least one exemplar recorded in the training mode for each intelligible-spell.

9. The device of claim 8, wherein the memory further comprises a database module retaining the user models; recorded exemplars for each spell; and at least one lexicon composed of the plurality of intelligible-spells.

10. The device of claim 8, wherein the memory further comprises a speech processing module utilized for determining, by user models stored in the database module, a matching intelligible-spell, stored in the database module, for each acquired probe, wherein said play the intelligible-spell is playing the matching intelligible-spell.

11. The device of claim 7, wherein said at least one speaker is configured for playing the intelligible-spell is playing a synthesized voice made of the text string of a matching intelligible-spell or a recording of the matching intelligible spoken words, wherein the text string is synthesized by the processor.

12. The device of claim 6, wherein the proxy computer, hosting a user's interface application, is configured as:
a text string data entry tool for entering spells or select spells from a predefined menu;
a sensitizer configured to make a synthesize voice from the text string of the intelligible-spell and a speaker that can play the synthesized voice;
a microphone adapted to acquire intelligible spoken words;
a platform for building a lexicon of spells to be grouped for different scenarios;
a display providing a visual representation of a waveform representing exemplars recorded in the training mode;
an editor for editing recorded exemplars; and
a proxy to the internet.

13. A speech interpretation system comprising:
the device of claim 2;
a proxy computer hosting a user's interface application configured as:
a text string data entry tool for entering spells or select spells from a predefined menu:
a sensitizer configured to make a synthesize voice from the text string of the intelligible-spell and a speaker that can play the synthesized voice;
a microphone adapted to acquire intelligible spoken words;
a platform for building a lexicon of spells to be grouped for different scenarios;
a display providing a visual representation of a waveform representing exemplars recorded in the training mode;
an editor for editing recorded exemplars; and
a proxy to the internet; and
a cloud computing server (CCS) comprising:
a speech recognition server;
a speech pattern recognition application; and
a speech recognition database,
wherein the computer and the CCS comprising communication interfaces for communicating with each other and the device.

14. The speech interpretation system of claim 13, wherein the communication interfaces are selected from a group consisting of: Wi-Fi interfaces; a Bluetooth interfaces; and USB interface, wherein the Bluetooth interfaces and the USB interface are configured for communicating between the proxy computer and the device, and wherein the Wi-Fi interfaces are configured for communicating between the device the proxy computer and the CCS via the Internet.

15. The speech interpretation system of claim 14, wherein the speech recognition database retains at least one exemplar recorded for each spell in the training mode; and at least one lexicon composed of the plurality of intelligible-spells obtained from the device in the training mode.

16. The speech interpretation system of claim 15, wherein the speech recognition server and the speech pattern recognition application generate a user model for each intelligible-spell of the plurality of intelligible-spells based on at least one exemplar recorded in the training mode for each intelligible-spell, and wherein the speech recognition server retains said user model for each intelligible-spell of the plurality of intelligible-spells in the speech recognition database and the database module of the device.

17. The speech interpretation system of claim 16, wherein the system is configured to concurrently communicate and manage and support a plurality of devices used by different registered users.

18. The speech interpretation system of claim 17, wherein said communicate manage and support comprising: user registration; access control; management registration credentials; users lexicon maintenance and user models updates for the different registered user.

19. A training mode method for the device of claim 12 comprising:
entering at least one intelligible-spell;
recording at least one exemplar for each intelligible-spell;
processing the at least one exemplar;
generating a user model for each intelligible-spell; and
retaining the user model in the database module.

20. The training mode method of claim 19, wherein said entering at least one intelligible-spell is selected from a group consisting of: typing a text string representation of the intelligible-spell by the computer; recording intelligible spoken words by the device or the computer; and a combination thereof.

21. (canceled)

22. A training mode method for the system of claim 13 comprising:
entering, by the proxy computer, one or more intelligible-spells;
recording, by the device, at least one exemplar for each intelligible-spell;
retrieving, by the speech recognition server, said one or more intelligible-spells and the recording of said at least one exemplar for each intelligible-spell;
processing the recording by the speech recognition server;
generating, by a speech pattern recognition application, a user model for each intelligible-spell; and
storing the user model in the speech recognition database and the database module of the device.

23-29. (canceled)
